(19) United States
(12) Patent Application Publication
(10) Pub. No.: US 2018/0133245 A1
(43) Pub. Date: May 17, 2018

(54) Impact of electrolytes on tumors and various diseases

(71) Applicant: Tatiana Balazs-keithley, Keller, TX (US)

(72) Inventor: Tatiana Balazs-keithley, Keller, TX (US)

(73) Assignee: Tatiana Balazs-keithley, Keller, TX (US)

(21) Appl. No.: 15/449,807

(22) Filed: Mar. 3, 2017

Related U.S. Application Data
(60) Provisional application No. 62/497,267, filed on Nov. 15, 2016.

(51) Int. Cl. A61K 33/00; A61K 9/00; A61Q 19/08; A61K 8/20; A61K 45/00

(52) U.S. Cl. CPC A61K 33/00; A61K 9/0019; A61K 2800/91; A61K 8/20; A61K 45/00; A61Q 19/08

(57) ABSTRACT

I am filing 8 claims, 7 of them are based on the impact received from electrolytes. At the moment I feel that this is a gray area with heavy regulations which are preventing people from receiving enormous benefits from I.V. fluids for conditions that are being treated with surgical interventions and/or medications that can have severe side effects. The methods I am claiming are side effect free and open to any age and gender. Because of the nature of electrolytes this is a holistic solution to heavy health and medical conditions. The same method will work to help with aging appearances. Claim number 8 using antibiotics to lower high blood pressure is an important discovery because most of the current blood pressure medications can have severe side effects which can trigger other heath problems.

Procedure for relief of difficulty swallowing for patients with various deceases: Intravenous injection of saline fluids on 150 pump speeds, the relief suppose to be within an hour or couple of hours, maybe sooner. Results might vary depends on a condition and [or] age. The treatment is open to any age and gender, clinical study needed.

Procedure for diminishing of thyroid nodule, Possibility of tumor treatment: Intravenous injection of saline fluids on 150 pump speeds, repeat every 6 to 7 days until it goes away. Do not combine with any other medication. The treatment is open to any age and gender, clinical study needed. Possibility of tumor treatment

Procedure of relief of spasmodic occurrences in the forehead and eye area Intravenous injection of saline fluids for about 2.5 hours. The result might be immediate or within a week, clinical study needed. It also works as a cosmetic procedure, smooth out wrinkles.

Procedure for a relief of a devastating side effects of haloperidol, narcotics overdose and allergic reactions on medications:

Intravenous injection of saline fluids, about 300 pump speed, repeat as needed, preferably once in a 3 days depending on a severity of condition, saline fluids give best results from every 3-4 days to a week. Open to any age and gender, clinical study needed

Use antibiotics in lowering high blood pressure due to internal inflammation, clinical study needed

1. I am claiming a method that will help break down tumors without surgical intervention.

2. I am claiming a method that will help diminish the side effects of Haloperidol.

3. I am claiming a method that will diminish the effects of a narcotic substance.

4. I am claiming a method that will relieve difficulty swallowing which can occur in various health conditions.

5. 1 am claiming a method that will help break down thyroid nodules without surgical intervention.

6. I am claiming a method to help with aging appearances.

7. I am claiming the use of antibiotics for lowering high blood pressure caused by internal inflammation.

8. I am claiming a method that will relieve forehead and eye spasms along with helping ophthalmology problems.
